(19) United States
(12) Patent Application Publication
(10) Pub. No.: US 2012/0215232 A1
(43) Pub. Date: Aug. 23, 2012

(54) SURGICAL FASTENER AND ASSOCIATED SYSTEMS AND METHODS

(76) Inventors: Russell G. Olsen, Cedar City, UT (US); Scott E. Bulloch, St. George, UT (US)

(21) Appl. No.: 13/400,564

(22) Filed: Feb. 20, 2012

Related U.S. Application Data
(60) Provisional application No. 61/444,555, filed on Feb. 18, 2011.
(60) Provisional application No. 61/446,471, filed on Feb. 24, 2011.

(51) Int. Cl. A61B 17/10

(52) U.S. Cl. 606/139

(57) ABSTRACT

According to one embodiment, an apparatus for installing and removing a fastener includes a handle and a shank coupled to the handle. The shank includes an external periphery, an interior channel, and at least one fastener engagement element at least partially positionable within the interior channel. The apparatus further includes a wire positioned within the interior channel and an actuator coupled to the wire. The actuator is actuatable to move the wire within the interior channel and into contact with the at least one fastener engagement element to move the at least one fastener engagement element into a fastener engagement position. In the fastener engagement position, at least a portion of the at least one fastener engagement element is positioned external to the external periphery of the shank.

### CROSS-REFERENCE TO RELATED APPLICATIONS

This application claims the benefit of U.S. Provisional Patent Application No. 61/444,555, filed Feb. 18, 2011, and U.S. Provisional Patent Application No. 61/446,471, filed Feb. 24, 2011, which are incorporated herein by reference.

### FIELD

This invention relates to fasteners and associated fastening devices and more particularly relates to surgical fasteners and associated installation and removal tools.

### BACKGROUND

Specialized fasteners for surgical (e.g., medical) applications are known in the art. In typical surgical applications, these specialized fasteners (e.g., screws) are fastened to the tissue (e.g., bone tissue) of a patient. Surgical fasteners are often used with other devices, such as pins, braces, and plates, in the setting and immobilization of bone fractures, as well as in other applications. Often, conventional surgical fasteners are fastened to the tissue by forming a hole in the tissue at a placement site and threading the fastener into the hole. This procedure commonly requires a medical professional performing the procedure to position the fastener proximate the placement site with one hand and with the other hand articulating an installation tool to drive the fastener into the hole in the tissue. Handling the fastener separately from the installation tool occupies both of the medical professional's hands and can be burdensome, awkward, and difficult to maintain a grip on and accurately place the fastener. Additionally, directly manually handling the fastener can increase the likelihood of harmful germs and bacteria transferring from the medical professional to the fastener prior to insertion into the tissue.

Some conventional fasteners employ various head and installation tool receptacle designs in an attempt to improve the coupling between the fastener and installation tool, which can improve the process of installing and removing fastener. However, such conventional fasteners often fail to provide adequate coupling between the fastener and installation tool for both installation and removal of a fastener, particularly where one-handed operation in medical and surgical applications is desired. Some systems include installation tools that secure the fastener to the installation tool prior to installation and removal in an attempt to facilitate one-handed operation. These systems, however, fail to provide adequate ease in operation and robustness necessary for many medical applications, as well as suffer from other significant shortcomings.

One common device used with surgical fasteners is a locking plate designed to internally fixate surgeon-induced bone fractures (e.g., for repair of a surgical osteotomy bone cut or fracture) or accidental bone fractures. Typical locking plates include a plurality of apertures configured to receive a respective one of a plurality of surgical fasteners. A threaded shank of each surgical fasteners is passed through an aperture of the locking plate and fastened to the tissue by forming a hole in the tissue at a placement site and threading the fastener into the hole. The apertures of the locking plate can include internal threads configured to threadably mate with external threads formed on the heads of the fasteners. Threadable engagement between the threads of the apertures and fastener head promotes stabilization between the plate and tissue should one or more of the threaded shanks of the fasteners fail to adequately engage the tissue.

Generally, installing the fasteners into tissue to secure a locking plate to the tissue is a relatively easy and uncomplicated process. An engagement portion of an installation tool is inserted into a recess in the fastener head and the tool is rotated in a tightening direction to secure the fastener relative to the tissue and locking plate. However, removing fasteners from engagement with the tissue and plate as part of a locking plate removal process can be quite difficult and complex. For example, while the fasteners and locking plate are secured to the tissue during a tissue healing period, the tissue tends to form about the fasteners and plate to bind the threads of the fastener, thus severely resisting removal. Also, over time, the fastener heads tend to bind with the plates to resist rotation of the fastener relative to the plate. Often, as a medical professional attempts removal of the fasteners by inserting the engagement end of the installation tool into the recess in the head and rotating the tool in a loosening direction, rotational resistance between the fastener, tissue, and locking plate causes the user to over-torque the tool, which often results in the tool stripping the recess in the fastener head. Fasteners with stripped fastener recesses require complicated and time-consuming procedures for removal.

Another problem often encountered during the installation of fasteners to secure a locking plate to tissue is the tendency of the fasteners to be misaligned as they are being inserted into the tissue. Also, during the formation of holes in the tissue preparatory to receiving the fasteners, the hole-forming drill has a tendency to be misaligned, thus forming misaligned holes in the tissue.

Other challenges analogous to those discussed above may also exist in non-medical fastener applications, such as applications involving materials (e.g., wood, metal, and plastic), or any applications where a reliable, easily operable, and secure fastener, and/or system and method for insertion and/or removal of the fastener is desired.

### SUMMARY

From the foregoing discussion, it should be apparent that a need exists for an apparatus, system, and method for the installation of surgical fasteners that promotes a secure engagement between an installation tool and the fasteners during transit from a sterile location to an installation site. Further, it should be apparent that a need exists for an apparatus, system, and method for the installation of surgical fasteners and locking plates that promotes ease in the installation of the fasteners and plates to tissue and the removal of the fasteners and plates from the tissue. Beneficially, such an apparatus, system, and method would also be useful in non-medical applications. The present subject matter has been developed in response to the present state of the art, and in particular, in response to the problems and needs in the art that have not yet been fully solved by currently available installation and/or removal tools, fasteners, and locking plates. Accordingly, the present subject matter has been developed to provide an apparatus, system, and method for installing fasteners and/or locking plates, as well as removing fasteners and/or locking plates, which overcome at least one, many, or all of the above-discussed shortcomings in the art.

According to one embodiment, an apparatus for installing and removing a fastener includes a handle and a shank coupled to the handle. The shank includes an external periphery, an interior channel, and at least one fastener engagement element at least partially positionable within the interior channel. The apparatus further includes a wire positioned within the interior channel and an actuator coupled to the wire. The actuator is actuatable to move the wire within the interior channel and into contact with the at least one fastener engagement element to move the at least one fastener engagement element into a fastener engagement position. In the fastener engagement position, at least a portion of the at least one fastener engagement element is positioned external to the external periphery of the shank.

In certain implementations, the shank includes a fastener engagement portion that includes at least one opening in the external periphery. In the fastener engagement position, the at least one fastener engagement element extends partially through the at least one opening. The fastener engagement portion of the shank is engageable with the fastener to form a first coupling between the shank and fastener. In the fastener engagement position, the at least one fastener engagement element is engageable with the fastener to form a second coupling between the shank and the fastener.

According to one implementation, the handle is graspable by a single hand of a user and the actuator is actuatable by said single hand while grasping the handle with said single hand.

In some implementations, the distal end portion of the wire includes a beveled surface. The actuator can be actuatable to move the wire to contact the at least one fastener engagement element with the beveled surface. The beveled surface promotes outward movement of the at least one fastener engagement element away from the interior channel.

According to some implementations, the at least one fastener engagement element includes at least one of a ball and pin. In some implementations, the at least one fastener engagement element includes a plurality of fastener engagement elements each separately movable relative to each other.

In one implementation, the actuator includes a plunging mechanism, which can include a depressible button. The depressible button is depressible a first time to move the wire within the interior channel and into contact with the at least one fastener engagement element to move the at least one fastener engagement element into the fastener engagement position. Further, the depressible button is depressible a second time to move the wire within the interior channel out of contact with the at least one fastener engagement element to allow the at least one fastener engagement element to move out of the fastener engagement position.

In yet one implementation, the actuator includes a collar that is translationally movable between first and second positions. In the first position, the wire is in contact with the at least one fastener engagement element to move the at least one fastener engagement element into the fastener engagement position. In the second position, the wire is out of contact with the at least one fastener engagement element to allow the at least one fastener engagement element to move out of the fastener engagement position, the collar being biased in the first position. The collar can be rotationally movable relative to the handle to rotate the shank relative to the handle.

According to one implementation, the actuator includes a swivel rotationally movable relative to the handle to translationally move the wire between first and second positions. In the first position, the wire is in contact with the at least one fastener engagement element to move the at least one fastener engagement element into the fastener engagement position. In the second position, the wire is out of contact with the at least one fastener engagement element to allow the at least one fastener engagement element to move out of the fastener engagement position.

In some implementations, the shank is a first internal shank. The apparatus further includes a second external shank. The first internal shank is positioned within the second external shank, and the second external shank comprising a plurality of teeth configured to engage the fastener. The actuator can be a first actuator, and the apparatus can include a second actuator coupled to the shank. The second actuator is actuatable to translationally move the second external shank relative to the first internal shank.

According to one embodiment, a fastener includes a head that includes a recess for receiving a first portion of a tool. The recess includes at least one sidewall and at least one indentation formed in the sidewall. The at least one indentation is configured to receive a second portion of the tool. The fastener also includes a shank coupled to the head where the shank includes external threads. The at least one indentation can include a plurality of substantially semi-spherically shaped indentations. The at least one indentation can be a single slot extending about the recess. The fastener can also include a plurality of notches formed in the head and positioned about the recess. In some implementations, the head includes a plurality of spaced-apart radially outwardly extending arms configured to radially inwardly collapse under a predetermined pressure threshold. An outer periphery of each of the arms includes external threads.

According to yet another embodiment, a system for installing and removing fasteners includes a tool that includes a handle and a driver portion coupled to the handle. The driver portion includes a first fastener engaging element, a shaft translationally movable relative to the handle, and a plurality of second fastener engaging elements outwardly movable in a direction substantially transverse relative to the translational movement of the shaft. The shaft is translationally movable via actuation of an actuator to contact and outwardly move the plurality of second fastener engaging elements. The system also includes a fastener that includes a head and a shank. The shank can extend substantially transversely relative to the head. The head includes a recess configured to matingly receive the first fastener engaging element of the tool. The recess includes at least one sidewall and at least one indentation formed in the at least one sidewall. The at least one indentation is configured to receive the plurality of second fastener engaging elements when outwardly moved by the shaft.

In one implementation of the system, the driver portion includes a plurality of third fastener engaging elements positionable about the first and second fastener engaging elements. The fastener may also include a plurality of notches positioned about the recess. The plurality of notches is configured to matingly receive the plurality of third fastener engaging elements.

In another embodiment, a method for installing fasteners into an object and removing fasteners from an object includes positioning a first fastener engaging element of a tool into a recess of a fastener. With the first fastener engaging element positioned in the recess of the fastener, the method includes actuating an actuator of the tool to outwardly move and retain at least one second engaging element of the tool into a corresponding at least one engaging element receiver formed in a sidewall of the recess. Positioning the first fastener engaging element and actuating the actuator of the tool can be performable with one hand.

In some implementations, with the first fastener engaging element in the recess of the fastener and the at least one second engaging element outwardly moved in the corresponding at least one engaging element receiver, the method includes positioning the tool proximate a fastener installation site on the object and rotating the tool in a driving direction to drive the fastener into the object. Also, with the fastener driven into the object, the method may include actuating the actuator of the tool to release the at least one second engaging element from the at least one engaging element receiver. Additionally, with the at least one second engaging element released from the at least one engaging element receiver, the method includes removing the first fastener engaging element of the tool out of the recess of the fastener.

According to certain implementations of the method, the at least one engaging element receiver includes at least one first engaging element receiver. The method further includes actuating a second actuator of the tool to move at least one third engaging element of the tool into a corresponding at least one second engaging element receiver formed in the fastener when the fastener is driven in an object. With the at least one third engaging element of the tool moved into the corresponding at least one second engaging element receiver formed in the fastener, and with the fastener installed in the object, the method includes rotating the tool in a loosening direction.

According to another embodiment, a locking plate system includes a locking plate that has a plurality of circular apertures with internal threads. At least one of the apertures includes a plurality of spaced-apart notches positioned about a periphery of the at least one of the apertures. The plurality of spaced-apart notches defines a plurality of spaced-apart tabs each positioned between adjacent notches. The internal threads of the at least one of the apertures are formed in the plurality of spaced-apart tabs.

In some implementations, the locking plate may further include circumferentially extending slits formed in the plurality of spaced-apart tabs. In yet some implementations, the locking plate further includes a screw alignment guide that has a threaded channel for receiving a threaded fastener. The alignment guide further includes a plurality of tabs sized, shaped, and positioned to matingly engage the plurality of spaced-apart notches of the at least one of the apertures and orient the threaded channel into coaxial alignment with the at least one of the apertures. The locking plate may also include a drill guide that includes a channel sized to matingly receive a drill bit. The drill guide is positionable within and in coaxial alignment with the threaded channel of the screw alignment guide.

Reference throughout this specification to features, advantages, or similar language does not imply that all of the features and advantages that may be realized with the subject matter of the present disclosure should be or are in any single embodiment or implementation of the subject matter. Rather, language referring to the features and advantages is understood to mean that a specific feature, advantage, or characteristic described in connection with an embodiment is included in at least one embodiment of the subject matter of the present disclosure. Discussion of the features and advantages, and similar language, throughout this specification may, but do not necessarily, refer to the same embodiment or implementation.

The described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### BRIEF DESCRIPTION OF THE DRAWINGS

In order that the advantages of the subject matter will be readily understood, a more particular description of the subject matter briefly described above will be rendered by reference to specific embodiments that are illustrated in the appended drawings. Understanding that these drawings depict only certain illustrative embodiments and are not therefore to be considered to be limiting of its scope, further embodiments of the subject matter will be described and explained with additional specificity and detail through the use of the drawings, in which:

FIG. 1 is a partial cross-sectional side view of a fastener installation and removal tool according to one embodiment;

FIG. 2 is a cross-sectional bottom view of a shank of the fastener installation and removal tool of FIG. 1 taken along the line 2-2 of FIG. 1.

FIG. 3 is a partial cross-sectional side view of a shank of a fastener installation and removal tool according to another embodiment;

FIG. 4 is a perspective top view of a fastener according to one embodiment;

FIG. 5 is a perspective top view of a fastener according to another embodiment;

FIG. 6 is a side view of a fastener installation and removal tool according to another embodiment with some internal features shown with hidden lines;

FIG. 7 is a side view of a fastener installation and removal tool according to yet another embodiment with some internal features shown with hidden lines;

FIG. 8 is a side view of a wire with a beveled end surface in contact with an engagement element according to one embodiment;

FIG. 9 is a cross-sectional side view of a cannulated wire with a beveled end surface according to one embodiment;

FIG. 10 is a cross-sectional side view of a fastener installation and removal tool according to a further embodiment;

FIG. 11 is a side view of a fastener installation and removal tool according to another embodiment;

FIG. 12 is a bottom view of the fastener installation and removal tool of FIG. 11;

FIG. 13 is a detailed side view of a driver portion of the tool of FIG. 11;

FIG. 14 is a top view of a fastener according to one embodiment;

FIG. 15 is a cross-sectional side view of a fastener installation and removal tool according to yet another embodiment;

FIG. 16 is a cross-sectional side view of a fastener installation and removal tool according to an additional embodiment;

FIG. 17 is a top view of a locking plate according to one embodiment;

FIG. 18 is a perspective top view of a fastener alignment guide according to one embodiment;

FIG. 19 is a perspective top view of a drill guide according to one embodiment;

FIG. 20 is a top view of a collapsible fastener engaged with an aperture of a locking plate according to one embodiment; and

FIG. 21 is a top view of the collapsible fastener of FIG. 20 shown in a collapsed state according to one embodiment.

### DETAILED DESCRIPTION

Reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the subject matter of the present disclosure, however, absent an express correlation to indicate otherwise, an implementation may be associated with one or more embodiments.

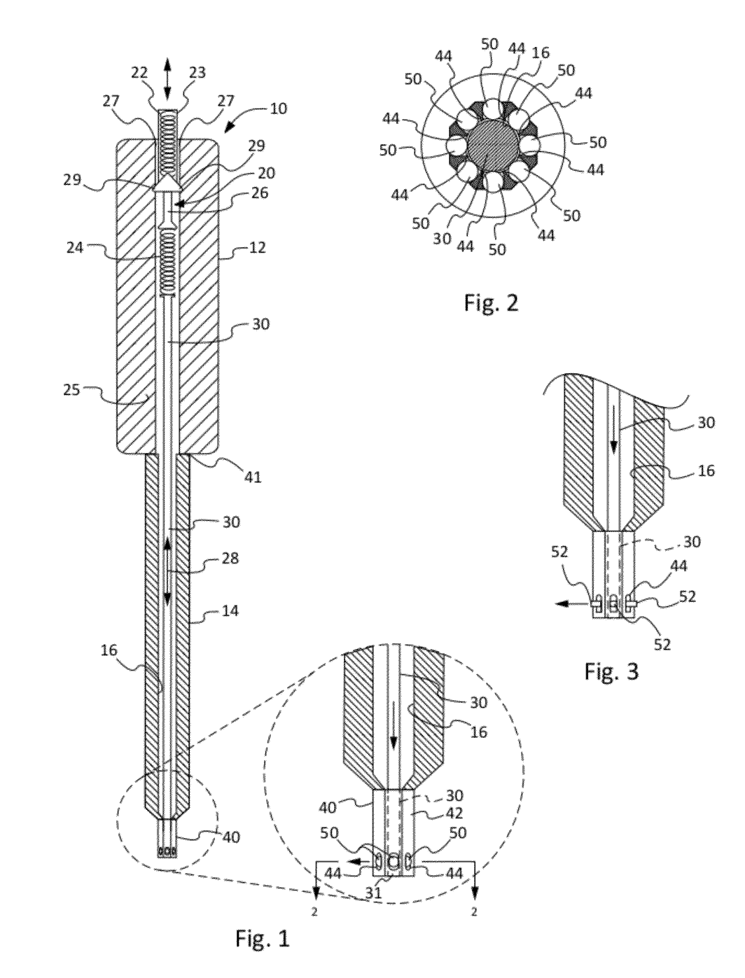

Referring to FIG. 1, and according to one embodiment, a fastener installation and removal tool 10 is shown that includes a handle 12 and a cannulated shank 14 having an external periphery and defining a hollow interior channel 16 along an internal periphery of the shank. The handle 12 includes an actuator, e.g., a plunging mechanism 20, configured to drive a shaft or wire 30 down through the hollow interior channel 16 in a first direction and retract the shaft up through the hollow interior channel in a second direction opposite the first direction as indicated by directional arrows 28. The plunging mechanism 20 includes two springs 22, 24. The first spring 22 biases an upper button 23 in a first position and the second spring 24 biases a piston 26 in a second position. The handle 12 defines a central channel 25 within which the springs 22, 24, upper button 23, and piston 26 are at least partially retained. Further, the handle 12 includes a series of axially-extending splines 27 positioned within the central channel 25. The spline 27 engage corresponding splines 29 formed in the upper button 23. As the upper button 23 is pressed down (e.g., depressed) and released relative to the handle 12, the splines 27, 29 disengage and reengage each other to alternate the position of the piston 26 between raised and lowered positions. Generally, in some implementations, the plunging mechanism 20 is similar to a conventional plunging mechanism for a retractable ball-point pen. The piston 26 is fixedly coupled to a proximal end of the shaft 30. Accordingly, movement of the piston 26 in the directions 28 correspondingly moves the shaft 30 in the same directions 28. For example, when the piston 26 is in the raised position, the shaft 30 is in a retracted position and when the upper button 23 is depressed and released to move the piston into the lowered position, the shaft 30 is in an extended position (see, e.g., FIG. 1).

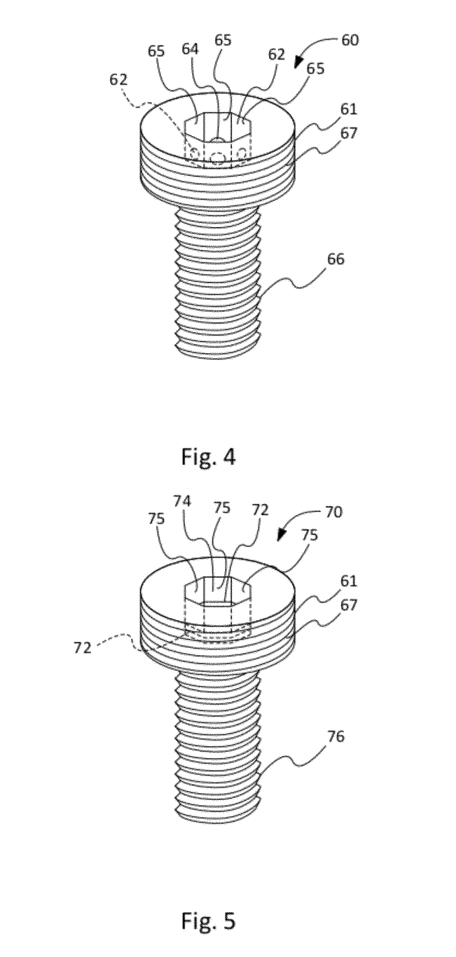

Referring to FIG. 1, a distal end 40 of the shank opposite a proximal end 41 of the shank coupled to the handle 12 includes a fastener head or recess engagement portion 42 shaped to be nestably inserted into a recess formed in the head of a fastener (see, e.g., the fasteners 60, 70 shown in FIGS. 4 and 5). The fastener head engagement portion 42 can be shaped to matingly receive (e.g., be matingly inserted into) any of various fastener head recess types (e.g., hexagonal, square, triangular, etc.). Further, the fastener head engagement portion 42 includes a plurality of slots or apertures 44 extending radially outward from the interior channel 16 (see, e.g., FIG. 2). The fastener head engagement portion 42 also includes one or more fastener engagement elements (e.g., balls 50 and/or pins 52) movably retained at least partially within the interior channel 16 and the slots 44. More specifically, the slots 44 formed in the fastener head engagement portion 42 engagement elements are sized to retain the engagement elements within the slots, but allow radially-directed movement 51 of the elements within the slots. In some implementations, the slots 44 include openings that open to the interior channel 16 and open externally to the head engagement portion 42, as well as a channel extending between the openings. The openings can be smaller than the engagement elements to retain the elements within the channel, and the channel can be large enough to allow movement (e.g., translational movement (e.g., slidable, linear, etc.)) within the channel.

As the plunging mechanism 20 is actuated (e.g., by pressing the upper button 23), the shaft 30 is driven downwardly within the interior channel 16. As the shaft 30 is driven downwardly, a distal end 31 of the shaft 30 will eventually contact the one or more engagement elements positioned adjacent the interior channel 16. Contact with the distal end 31 of the shaft 30 forces the engagement element(s) through a respective one of the plurality of slots or apertures 44 in a generally radially outward direction as indicated by directional arrow 51. The extended engagement elements extend through a respective one of the slots 44 such that a portion of the engagement elements extends radially outwardly beyond an outer or external periphery of the fastener head engagement portion 42. The plunging mechanism 20 can again be actuated to retract the distal end 31 of the shaft 30 upwardly within the interior channel 16 to move the distal end 31 out of engagement with the engagement elements. With the distal end 31 of the shaft 30 retracted away from the engagement elements, the engagement elements are free to move or retract radially inwardly through the slots or apertures 44 such that no portion of the engagement elements extend radially outwardly beyond the periphery of the fastener head engagement portion 42. The engagement element can be any of variously sized and shaped objects, such as a ball 50 (see, e.g., FIG. 1) or a pin 52 (see, e.g., FIG. 3). Although only one ball or pin is shown in FIGS. 1 and 3, in other embodiments, such as shown in FIG. 2, more than one ball or pin can be used as desired without departing from the essence of this disclosure.

Referring to FIGS. 4 and 5, fasteners 60, 70 according to the present disclosure each includes a head 61, 71 coupled to a threaded shank 66, 76, respectively. The heads 61, 71 include respective fastener head recesses 64, 74 formed in a top surface of the heads. The recesses 64, 74 are defined by one or more sidewalls 65, 75. The recesses 64, 74 are sized and shaped to matingly receive the head engagement portion 40 of the tool 10. For example, the illustrated recesses 64, 74 each has a substantially octagonal cross-sectional shape and the head engagement portion 40 has a substantially octagonal cross-sectional shape sized to be matingly inserted into the recesses. Generally, the mating engagement between the head engagement portion 40 and the recesses 64, 74 provides a non-interference fit such that the head engagement portion is easily insertable into and removable from the recesses. Although octagonal-shaped head engagement portion and recesses are shown, in other embodiments the head engagement portion and recess can have any of various, preferably non-round, shapes and sizes.

Further, the heads 61, 71 include engagement element receivers 62, 72, respectively, formed in the sidewalls 65, 75 of the fastener head recesses 64, 74, respectively. The engagement element receivers 62, 72 are configured to engage the engagement elements when the elements are pushed radially outward by the shaft 30 as the shaft is driven downwardly via actuation of the plunging mechanism. For example, the fastener head 61 of FIG. 4 includes semi-circular, semi-spherical, or rounded engagement element receivers 62 (e.g., indentations) formed in the fastener head recess 64 to matingly engage (e.g., receive) the ball or balls 50 when forced radially outward. Alternatively, the fastener head 71 of FIG. 5 includes a plurality of slot-shaped engagement element receivers 72 (e.g., indentations) or a single annular-ring-shaped engagement element receiver 72 (e.g., indentations) formed in the recess 74 to engage the pin or pins 52 when forced radially outward. It is noted that existing fasteners with any of variously shaped recesses can be modified to include engagement element receivers such as those described herein to accommodate the use of the tool 10 with existing fasteners.

Although some of the fastener tools shown include multiple fastener engagement elements (e.g., eight in tool 10 and four in tool 252), in some implementations, the tool only includes one engagement element. Moreover, the shanks of the tools include one slot for each engagement element. Accordingly, for tools with a single engagement element, the shank includes only one slot through which the engagement element extends. The larger the fastener and tool, the more engagement elements can fit in the shank. However, for applications using very small fasteners and tools, the shank may only have room for one fastener engagement element. Despite a tool only having one fastener engagement element, the recess of the fastener can have one or multiple receivers for receiving the element. For implementations with a single engagement element and multiple receivers in the fastener, regardless of the orientation of the tool when engaged with the recess, the single engagement element will always be aligned with one of the multiple receivers to facilitate the second coupling.

The fastener tool (e.g., tool 10) and fasteners (e.g., fasteners 60, 70) of the present disclosure are configured to promote the temporary coupling and retaining of a fastener to the tool in a one-handed manner while the tool and fastener are moved into placement proximate a fastener installation site. In operation, and with reference to the fastener tool 10 and fastener 60, the fastener head engagement portion 40 of the tool is inserted into the recess 64 of the fastener head 61. The initial engagement between the fastener head engagement portion 40 and the recess 64 provides a first, relatively non-secure, coupling between the tool and fastener. For example, although the first coupling is secure when pressing the tool 10 and fastener 60 together to secure the fastener to an object, when the tool and fastener are not pressed together, the first coupling fails to retain engagement between the tool and fastener. Accordingly, the fastener tool 10 and fasteners 60 of the present disclosure provide a second coupling that temporarily retains the fastener in engagement with the tool when the tool and fastener are not pressed together, such as when a user is orienting or transporting the tool and fastener into position to secure the fastener to the object.

The second coupling is formed by actuating the plunger mechanism 20, or other wire actuation mechanism, as discussed above. More specifically, after forming the first coupling by inserting or positioning the tool 10 within the recess 64 of the fastener 60, the plunger mechanism 20 is actuated to drive the distal end 31 of the wire downwardly to force the engagement elements 50 radially outward into mating engagement with the engagement element receivers 62 of the recess 64. Engagement between the engagement elements 50 and the engagement element receivers 62 defines the second coupling, which at least temporarily couples and retains the fastener 60 to the fastener head engagement portion 40 of the tool 10 without pressing the fastener and tool together and regardless of the spatial orientation of the tool and faster. Accordingly, the second coupling allows a user to transport the installation tool with the engaged fastener from a sterile location (e.g., a surgical tray) to a fastener implantation site with one hand without the fastener becoming dislodged or disengaged with the tool and without the other gloved or non-gloved hand touching the fastener. For example, the first and second coupling can be accomplished with a single hand by gripping the tool with one hand, manipulating the position of the tool to engage the fastener to form the first coupling, and actuating the plunging mechanism with the thumb of the hand to form the second coupling. Accordingly, use of the installation tool and fastener described herein virtually eliminates the risk of hand-to-fastener contamination and external-object-to-fastener contamination prior to implantation or installation of the fastener in an object (e.g., bone tissue).

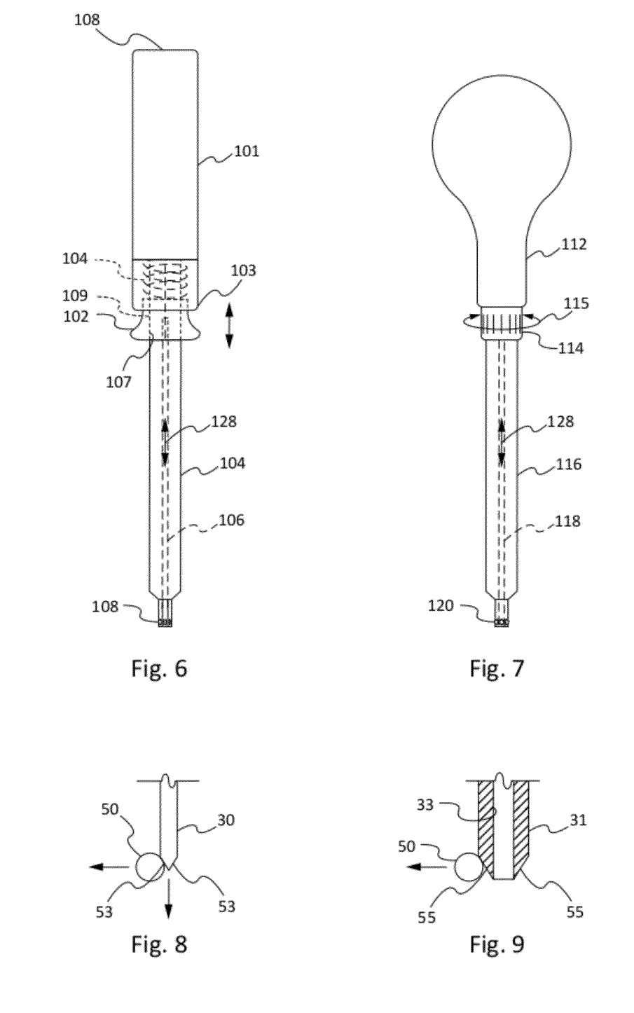

According to another embodiment shown in FIG. 6, an installation tool 100 includes a handle 101 and a cannulated shank 104 similar to tool 10. However, at a base 103 of the handle 101, away from a proximal end 105 of the handle, the installation tool 100 includes an actuator, e.g., collar 102, that is movably coupled to the handle 101. For example, the collar 102 includes a central channel 107 through which the shank 104 extends. The collar 102 can be slidably or translationally movable along the shank 104 in the directions indicated by directional arrow 129. Additionally, the collar 102 is fixedly secured to a shaft or wire 106 that is movable within a hollow interior of the shank 104. The collar 102 is movably coupled to the handle 101 via a biasing member or spring 109 that biases the collar in a first position corresponding with an extended position of the shaft 106. In the extended position, the shaft 106 engages an engagement element, such as the ball 108, to force the ball radially outwardly in the same manner as discussed above. To retract the shaft 106 and the ball into a retracted position, the collar is moved upward toward the handle against the biasing force of the spring.

In the retracted position, the shank can be inserted into an unengaged fastener or removed from an engaged fastener. Once the collar 102 is disengaged by the user, the bias of the spring urges the collar and wire back into the extended position. Accordingly, to engage and secure a fastener to the tool 100 with a single hand, the collar 102 can be moved (e.g., pulled) into the retracted position with a thumb or finger of the hand, the distal end of the shank can be positioned within a recess of the fastener, and the collar can then be released by moving the thumb or finger off of the collar such that the ball (or balls) is forced into engagement with a respective one of the engagement element receivers of the fastener. If desired, in some embodiments, the spring 109 can bias the collar 102 in an opposite direction to bias the shaft 106 in the retracted position. In such embodiments, to form the second coupling with a fastener, the user moves (e.g., pushes) the collar 102 with a thumb or finger to move the shaft 106 into the extended position.

FIG. 7 shows yet another embodiment of a tool 110 that is actuatable to move a shaft or wire 118 within a hollow shank 116 to force a ball 120 (or other engagement member) radially outwardly into engagement with an engagement element receivers of a fastener in a manner similar to the above-described embodiments. However, the tool 110 is actuatable by rotating an actuator, e.g., swivel 114, relative to a handle 112 as indicated by directional arrows 115. In one implementation, the swivel 114 and wire 118 are threadably engaged such that rotation of the swivel causes the threads of the swivel and wire to rotate relative to each other to translationally raise (e.g., extend) and lower (e.g., retract) the wire relative to the swivel, handle 112, and shank 116 for extending and retracting the ball 120. In another implementation, the swivel 114 and handle 112 are threadably engaged such that rotation of the swivel causes the swivel to translationally move relative to the handle, and thus translationally move the wire relative to the handle. Accordingly, the tool 110 facilitates one-handed operation by allowing a user to grasp the handle 112 with one hand and turn the swivel 114 with a thumb and finger of the same hand.

As shown in FIG. 8, in some embodiments the distal end of the shaft or wire (e.g., shaft 30 of the tool 10), which is shown as a solid wire, can include one or more beveled surfaces 53 to facilitate radially outward movement of the ball 50, pin 52, or other engagement element when contacted by the wire. Similarly, a cannulated wire 31 similar to wire 30, but with a hollow interior or channel 33, can include a distal end with one or more beveled surfaces 55 to facilitate radially outward movement of the ball 50, pin 52, or other engagement element when contacted by the cannulated wire. The cannulated wire 31 can be used in applications involving a cannulated fastener designed to be installed with the guidance of a guide wire.

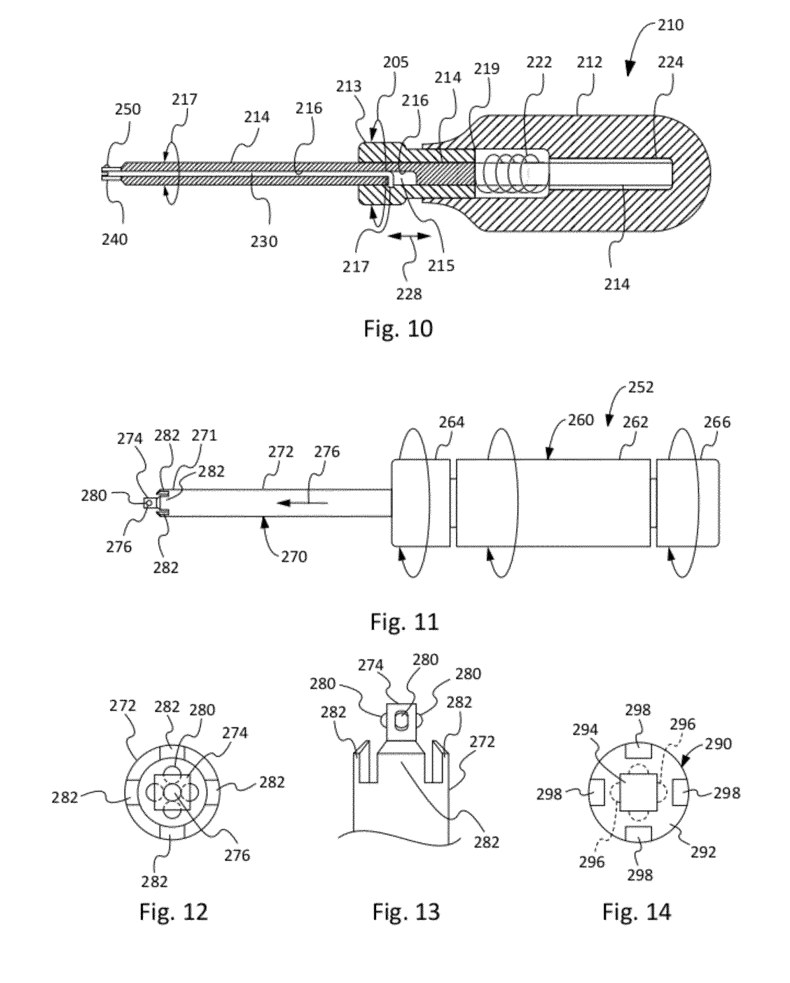

According to another embodiment shown in FIG. 10, a fastener installation tool 210 includes a handle 212 and a cannulated shank 214 defining an interior channel 216. At the base of the handle 212, away from a proximal end of the handle, the installation tool 210 includes an actuator, e.g., a collar or slide mechanism 213, fixedly secured to a wire 230 that is movable within a hollow interior of the shank 214. The wire 216 includes a laterally extending portion 217 that is engageable with (e.g., adhered to, bonded to, or press-fit into) the slide mechanism 213 to facilitate co-movement of the wire relative to the slide mechanism (e.g., fixed coupling between the wire and slide mechanism). The cannulated shank 214 may include a slot 215 to allow translational or linear movement (as indicated by directional arrows 228) of the portion 217 along the shank as the wire is extended and retracted.

Similar to the tool 100 of FIG. 6, the slide mechanism 213 is biased in a first position corresponding to an extended position of the wire 230 via a biasing member or spring 222. In the same manner as discussed above, in the extended position, the wire 230 (e.g., a distal end portion of the wire) engages an engagement element, such as a ball 50, proximate a distal end 240 of the shank 214 to force the ball radially outwardly to engage a corresponding indentation formed in a fastener head recess and secure the tool 210 to the fastener by forming a second coupling. To retract the wire 230 and ball 250 into a retracted position, the slide mechanism 213 is moved (e.g., pulled) upward toward the handle against the biasing force of the spring 222. In the retracted position, the shank can be removed from a fastener, which has been engaged with the shank via the ball. Once the slide mechanism 213 is disengaged by the user, the bias of the spring 222 urges the slide mechanism 213 and wire 230 back into the extended position. Accordingly, to engage and secure a fastener to the tool 210 with a single hand, the slide mechanism 213 can be moved into the retracted position, the distal end of the shank can be positioned within a recess of the fastener, and the collar can then be released such that the ball (or balls) is forced into engagement with a respective one of the engagement element receivers of the fastener.

In contrast to the tool 100, the slide mechanism 213 is also rotatable relative to the handle 212 as indicated by directional arrows 205. As the slide mechanism 213 is rotated, the wire 230 and shank 214 also are rotated. Simultaneous co-rotation between the slide mechanism 213, shank 214, and wire 230 as indicated by directional arrows 205, 217, and relative linear movement between the shank and wire as indicated by directional arrows 228, can be facilitated by engagement between corresponding splines or flat surfaces formed on the shank and interior channel 219 of the slide mechanism. The corresponding splines or flat surfaces engage each other to prevent relative rotation between the slide mechanism 213 and the shank. Additionally, to facilitate rotation of the shank 214 relative to the handle 212, a sleeve, bearing, or bushing 224 can be positioned around the shank within the handle (e.g., between the shank and the handle). In appropriate applications, such as oral applications, a user can not only engage and disengage a fastener with a single hand by pulling and releasing, respectively, the slide mechanism 213, but can also rotate the slide mechanism 213 relative to the handle 212 in a more precise manner (e.g., with the thumb and index finger of the hand holding the tool 210) to rotate and install a fastener in tissue.

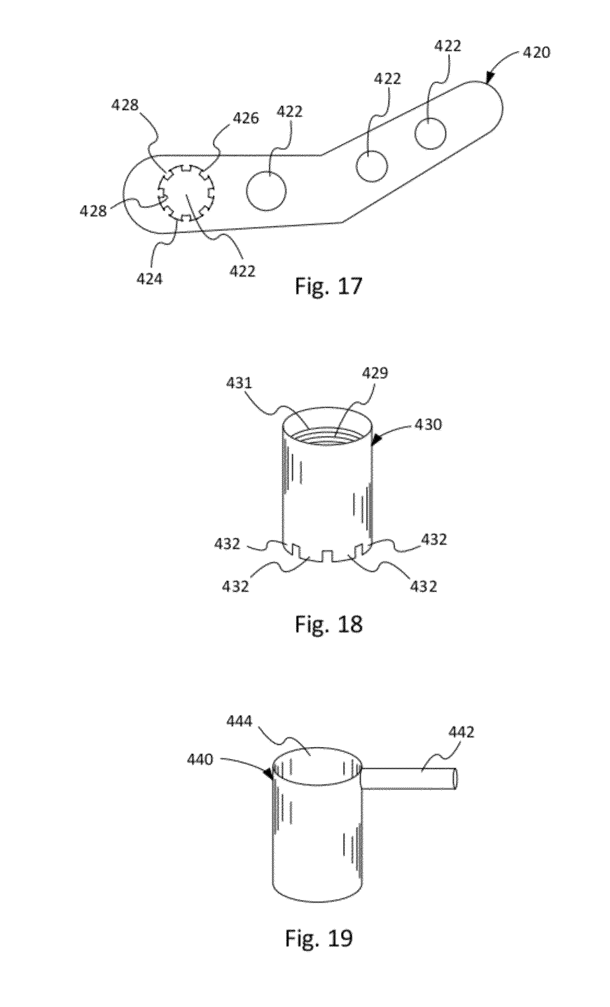

According to another embodiment, a tool 252 similar to the tool 210 of FIG. 10, but specifically configured to be used in a locking plate system (see, e.g., locking plate system shown in FIGS. 17-19) is shown in FIG. 11. Like the tool 210, the tool 252 includes an internal wire 276 that is movable relative to an internal hollow shank 274 of a driver portion 270 of the tool to force a ball 280 radially outwardly to engage a corresponding indentation formed in a fastener head recess and secure the tool to the fastener prior to insertion of the fastener. However, the tool 252 also includes features configured to facilitate removal of previously inserted fasteners, especially those fasteners that prove difficult for removal, such as fasteners that are bound or fused to tissue or locking plates, fasteners that are stripped, or fasteners that are mal-aligned relative to tissue or locking plates. For example, the driver portion 270 of the tool 252 includes an external hollow shank 272 within which the internal hollow shank 274 and wire 276 are positioned (also see, e.g., FIGS. 12 (bottom view) and 13 (side view)).

The external hollow shank 272 includes a plurality of teeth 282 positioned at a distal end 271 of the external shank in a spaced-apart manner about a periphery of the external shank. The teeth 282 are configured to be matingly received within corresponding notches formed in a head of a fastener (see, e.g., peripheral notches 298 of head 292 of fastener 290 shown in FIG. 5) to form a third coupling with the fastener. The internal hollow shank 274 is configured to be matingly received in a main recess or receptacle 294 formed in the fastener head 292. The recess 294 also includes a plurality of indentations 296 for receiving the one or more balls 280 of the tool when radially outwardly forced by the wire 276 (as shown, e.g., in FIGS. 12 and 13). Although the internal hollow shank 274 and recess 294 are shown to have a generally square cross-sectional shape, in other implementations, they can have any of various corresponding shapes. It is noted that existing fasteners with any of variously shaped heads can be modified to include peripheral notches such as those described above to accommodate the use of the tool 252 with existing fasteners. Also, although the tool 252 includes both components for facilitating the second and third couplings with a fastener, in some implementations, the tool 252 may include only those components for facilitating the first and third couplings, such that the components that facilitate the second coupling (e.g., the internal shank 274, wire 276, and ball 280) are not included with the tool.

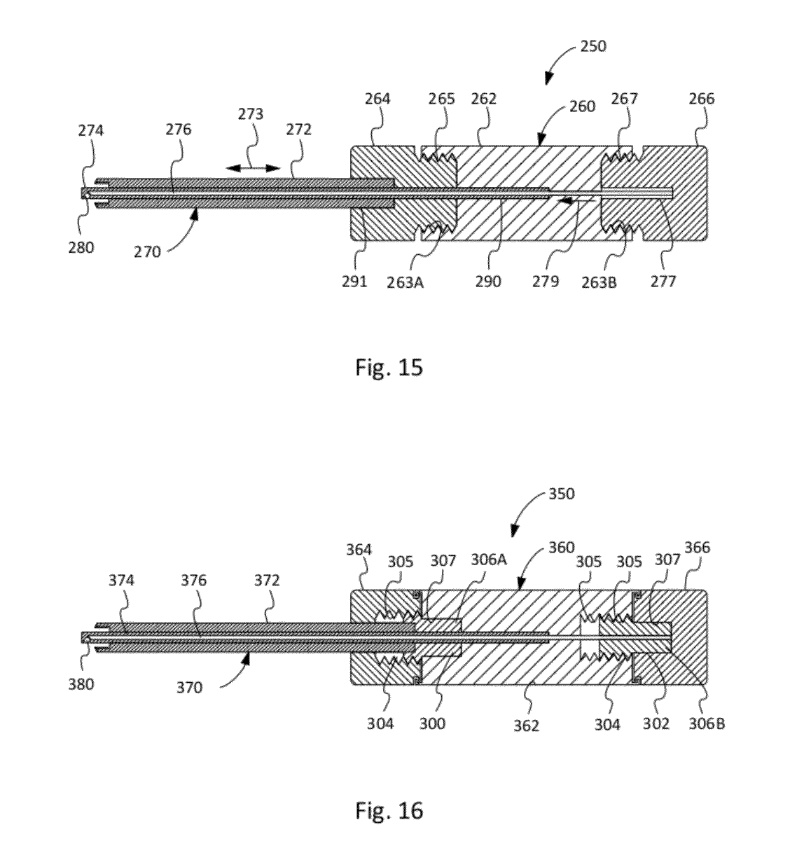

Referring back to FIG. 11, the tool 252 includes a handle 260 coupled to the drive portion 270. The handle 260 is used to grip the tool during a fastener insertion and/or removal procedure. Additionally, the handle 260 is operable (e.g., articulatable) to move the wire 276 up and down relative to the internal shank 274, and move the external shank 272 relative to the internal shank (see, e.g., FIG. 15). In one embodiment, the handle 260 includes at least one actuator, e.g., three interconnected sections 262, 264, 266, rotatable relative to each other.

According to the illustrated implementation in FIG. 11, the first section 264 is rotated in a first direction (e.g., a counterclockwise direction or fastener loosening direction) relative to the second section 262 to move the external shank 272 in a distal direction (e.g., away from the second section) relative to the internal shank 274 as indicated by directional arrow 273. Similarly, the third section 266 is rotated in the first direction relative to the second section 262 to move the wire 276 in the distal direction relative to the internal shank 274 as indicated by directional arrow 2799. Such an implementation is effective for gripping the entire handle 260 to remove an installed fastener because rotating any portion of the handle will not only rotate the fastener in the counterclockwise direction for removal, but will maintain the external shank 274 and wire 276 in an extended position during removal such that the tool's engagement with the fastener is not loosened.

In other implementations, the handle 260 can be configured such that the first section 264 is rotated in a second direction opposite the first direction (e.g., a clockwise direction or fastener tightening direction) relative to the second section 262 to move the external shank 272 in the distal direction relative to the internal shank 74. Also, the third section 266 can be rotated in the second clockwise direction to move the wire 276 in the distal direction relative to the internal shank 274. Such an implementation is effective when only the second or middle section 262 of the handle 260 is specifically configured for gripping during removal of an inserted fastener and the first and third sections 264, 266 are not used for gripping during removal. For example, the middle section 262 can have a significantly wider circumference than the first and third sections 264, 266, and/or have gripping features such as a rubber coating.

The first, second, and third sections 262, 264, 266 of the handle 260 can be interconnected and rotatable relative to each other in any of various ways. For example, as shown in FIG. 15, the first, second, and third sections 262, 264, 266 of the handle are interconnected via respective threaded connections. More specifically, the second or middle section 262 includes opposing internally threaded recesses or female connections 263A, 263B each configured to threadably receive respective externally threaded protrusions or male connections 265, 267 of the first and third sections 264, 266. A proximal end 277 of the wire 276 is fixedly coupled to the third section 266 such that as the third section is rotated relative to the second section 262, the third section moves linearly or translationally in an axial direction relative to the second section and the wire is driven relative to the second section and the internal shank 274, which is fixedly coupled to the second section at a proximal end 290. Similarly, a proximal end 291 of the external shank 272 is fixedly coupled to the first section 264 such that as the first section is rotated relative to the second section 262, the first section moves linearly or translationally in an axial direction relative to the second section and the external shank is driven relative to the second section and the internal shank 274.

FIG. 16 shows another embodiment of a tool 350 similar to the tool 250 of the embodiment of FIG. 15, with like numbers referring to like elements, but instead of the first and third sections 364, 366 moving linearly relative to the second section 362 as the first and third sections are rotated to drive the wire 376 and external shank 372, the tool 350 includes carriers 300, 302 that drive the external shank and wire, respectively, as the first and third sections are rotated such that the first and third sections do not move linearly or translationally relative to the second section, but simply rotate in place. The carriers 300, 302 include threaded portions 304 that threadably engage threaded portions 305 of the first and third sections 364, 366. Also the carriers 300, 302 include non-round portions 306 (e.g., a keyed, notched, or flat surface) corresponding to non-round recesses 307 formed in the first and third sections. Engagement between the non-round portions 306 and recesses 307 prevent co-rotation of the carriers 300, 302 with the first and third sections 364, 366, but allow linear or translational movement of the carriers relative to the first and third sections by virtue of the non-round portions 306 engaging the corresponding non-round recesses 307 in the second and third sections, respectively. It is noted that the tool 350 can be disengaged from a fastener by rotating the first and third sections 364, 366 in directions opposite the directions associated with engagement of the fastener as discussed above.

According to one method of using the tools 250, 350 described above, a user actuates the first section 264, 364 with the internal shank 274, 374 engaged to a main recess of a fastener to force the ball(s) radially outwardly to engage the indentation(s) of the main recess. Then, the user actuates the third section 266, 366 to drive the teeth of the external shank 272, 372 into engagement with the notches along the periphery of the head of the fastener. The user can then co-rotate the entire handle 260 of the tool, or just the middle section 262, 362 of the tool depending on the implementation, in a loosening direction to loosen and ultimately remove the fastener. Engagement between the teeth of the external shank and notches of the fastener dramatically increases the torque that can be applied to the fastener without stripping the main recess of the fastener with the internal shank. Accordingly, the combination of the engagement between the internal shank of the tool and the main recess of the fastener, and the engagement between the external shank of the tool and the notches of the fastener, facilitate effective removal of fasteners, particularly those that are essentially stuck in place.

In certain applications, a fastener is used to secure a locking plate, such as locking plate 420 of FIG. 17, to an object. The locking plate 420 has a plurality of circular apertures 422 each with internal threads 423 to engage the external threads of a fastener head (e.g., the external threads 67, 77 formed in the heads 61, 71 of the fasteners 60, 70 of FIGS. 4 and 5, respectively). The locking plate 420 is secured to an object, such as bone tissue, by positioning the apertures 422 of the locking plate over fastener installation sites, threading the shanks of the fasteners into the object, and threading the heads of the fasteners into respective threaded apertures of the locking plate. To facilitate the removal of fasteners, particularly severely stuck fasteners (e.g., when the threads of the fastener head have effectively bonded to the threads of the apertures in the plate), one or more of the apertures 422 may include a plurality of notches 424 about the periphery of the apertures. The notches 424 can be used to receive a prying tool in an attempt to pry the fastener head loose from the aperture 422 when the head is severely stuck. If prying does not work, in some implementations, the apertures 422 can be formed with relief slits or cuts 426 adjacent the base of the tabs 428 that define the notches 424 therebetween. Each slit 426 extends in a generally circumferential direction about a central axis of the respective aperture. The slits or cuts 426 weaken the tabs such that sufficient prying will break the tabs to effectively break the bond between the threads of the fastener head and aperture.

As shown in FIG. 18, to facilitate proper alignment between a fastener installation tool (e.g., the tools described above and/or a power drill), a fastener, and an aperture 422 of the locking plate 420 during a fastener insertion process, a locking plate system can include a screw or fastener alignment guide 430. The screw alignment guide 430 includes a central channel 429 with internal threads 431 that are mateable with the external threads of a screw head. Additionally, the screw alignment guide 430 includes teeth or tabs 432 that are sized, shaped, and positioned to mate with the notches 424 of the aperture 422 in the locking plate (see FIG. 17). The teeth 432 of the guide 430 can be placed in corresponding notches 424 of the aperture 422. Engagement between the teeth 432 and the notches 424 ensure proper rotational and vertical alignment between the aperture 422 and the central channel 429. Accordingly, as a fastener is threadably driven through the guide 430 and ultimately into the aperture 422 of the locking plate, the guide 430 effectively aligns the fastener with the aperture 422, and an installation hole pre-formed in the object to which the locking plate is being secured, as the fastener is being inserted into the locking plate aperture and the pre-formed hole.

Referring to FIG. 19, to ensure proper orientation of the pre-formed hole in the object (e.g., bone tissue), a locking plate system may include a drill guide 440 that is insertable into the channel 429 of the screw alignment guide 430 while the guide 430 is engaged with the notches 424 of the aperture 422. For ease in handling the drill guide 440, it may include a handle 442. The outer periphery of the drill guide 440 has a cross-sectional shape and size substantially similar to the channel 429, such that the channel 429 matingly engages the outer periphery of the drill guide as it is inserted into the channel to coaxially align the drill guide with the channel. After inserting the drill guide 440 into the screw alignment guide 430, a drill bit is then inserted into a channel 444 formed in the drill guide 440 and driven into the object or tissue. The channel 444 is sized and shaped to matingly receive and align the drill bit. Accordingly, the drill guide 440 aligns and guides the drill bit as the bit is driven into the tissue to form the hole in the tissue. In this manner, the proper or desired placement and orientation of the pre-formed hole in the object or tissue is ensured. After the hole is formed, the drill guide 440 can be removed from the screw alignment guide 430 using the handle 442, and the alignment guide 430 can be used to insert a fastener into the pre-formed hole and locking plate aperture. After the fastener is threadably engaged with the tissue and the aperture in the locking plate, the alignment guide 430 is simply lifted out of engagement with the notches of the locking plate aperture and removed.

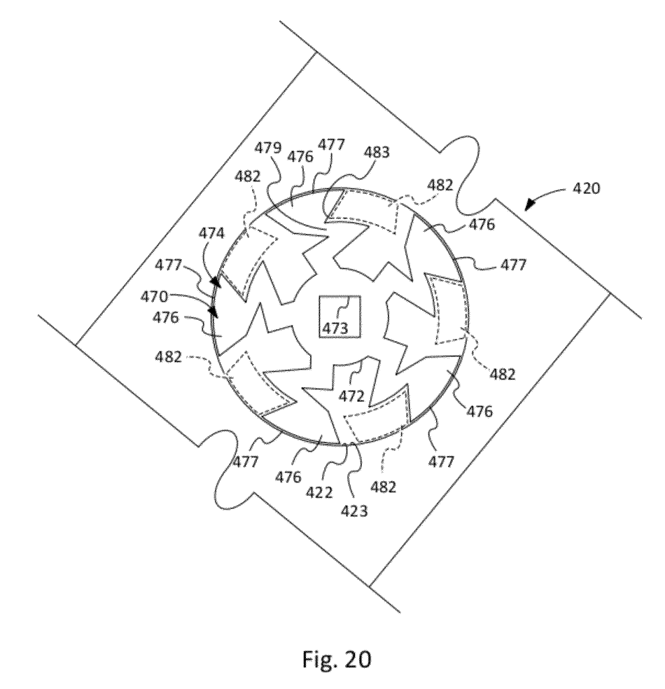

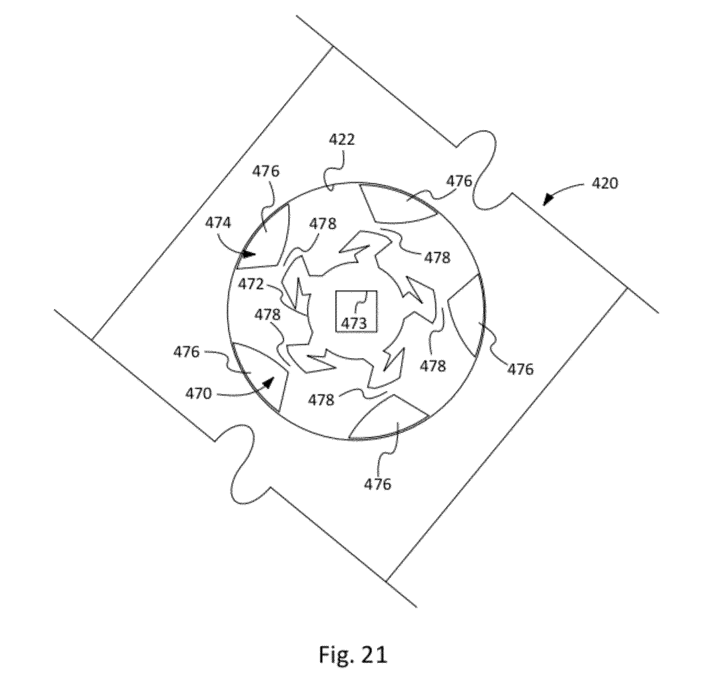

Referring to FIGS. 20 and 21, a fastener 470 according to one embodiment is shown with a fastener head 474 configured to deform to facilitate removal of the fastener from an aperture of a locking plate should the fastener head become stuck in the aperture (e.g., bond with the threads of the aperture). The fastener 470 includes the head 474, which includes a central hub 472 to which a plurality of collapsible arms 476 is coupled to and extends radially outwardly from the hub. The central hub 472 includes a recess 473 for receiving the fastener head engaging portion of an internal shank of a tool (e.g., fastener head engaging portion of internal shank 274 of tool 252). The central hub 472 is aligned with a shank (not shown) of the fastener 470 that extends into the page in the view shown in FIGS. 20 and 21. The shank includes external threads that engage an object, such as bone tissue, while the radially outer peripheries of the arms 476 of the fastener head 474 include corresponding external threads 477 that engage internal threads formed in the aperture 422 of the locking plate 420 to secure the locking plate to the head, which in turn secures the locking plate to the object.

The arms 476 are configured to collapse and/or shear under a predetermined pressure applied to the arms by a fastener removal tool. In some implementations, the fastener removal tool can be similar to the tools disclosed herein. In one specific implementation, the fastener removal tool can be similar to tool 252 where the teeth 282 of the external shank 272 are sized, shaped, and positioned to engage respective particular locations on the arms 476 to effectuate a collapsing or shearing of the arms. As shown in FIG. 20, the teeth 482 of a tool (shown in dashed lines) are positionable between the arms 476 such that a leading edge 483 of each tooth 482 is contactable with a weak link portion 479 of the arms. The weak link portion 479 of each arm is located at an intersection between a threaded, larger-massed, and radially-outer portion and a thinner, z-shaped, and radially-inner portion between the hub 472 and the outer portion.

As the tool is rotated in a loosening direction (e.g., counter-clockwise direction), the weak link portion 479 is configured to shear or allow the arm to collapse upon itself as a somewhat radially inwardly pressure applied to the weak link portion by the leading edge 483 exceeds a certain threshold. For example, referring to FIG. 21, the fastener 470 is shown in a collapsed or sheared state with the arms sheared at the weak link portion to separate the arms 476 at a location 478 into portions bonded to the aperture 422 of the locking plate 420 and collapsed portions coupled to the hub 472. Because the collapsed portions of the arms are physically separated from the bonded portions, the hub 472 and shank are effectively separated from the locking plate 420, which allows the locking plate to be easily removed from the fastener and the fastener to be removed from the object without resistance from the locking plate. Although the illustrated embodiment of FIG. 21 shows the arms 476 sheared into two portions, in other embodiments with weaker bonding between the head 474 and aperture 422, collapsing of the arms due to the pressure applied to the weak link portions 479 by the teeth 482 does not result in shearing of the arms. Rather, in such embodiments, the entire arms 476 collapse, including the portion of the arms engaged with the threads of the aperture 422, to separate the fastener 470 from the aperture.

The predetermined pressure threshold associated with the collapsibility of the arms 476 corresponds with a fastener head 474 that is significantly stuck or bonded to the threads of the aperture 422. Accordingly, if the fastener head 474 is not stuck or bonded to the threads such that a pressure less than the pressure threshold applied by the teeth 482 of the installation tool is sufficient to rotate the fastener relative to the aperture to remove the fastener, the arms 476 of the fastener 470 are configured to withstand the lesser pressure to resist collapsing and remain in an uncollapsed state as the fastener is removed from the aperture. Similarly, because the pressure to install the fastener into the aperture 422 will typically be less than the pressure threshold, the fastener remains in an uncollapsed state as the fastener is installed in the aperture using, for example, a tool with the teeth 482.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

The subject matter of the present disclosure may be embodied in other specific forms without departing from its spirit or essential characteristics. The described embodiments are to be considered in all respects only as illustrative and not restrictive. The scope of the subject matter of the present disclosure is, therefore, indicated by the appended claims rather than by the foregoing description. All changes which come within the meaning and range of equivalency of the claims are to be embraced within their scope.

What is claimed is:

1. An apparatus for installing and removing a fastener, comprising:
a handle;
a shank coupled to the handle, the shank comprising an external periphery, an interior channel, and at least one fastener engagement element at least partially positionable within the interior channel;
a wire positioned within the interior channel; and
an actuator coupled to the wire, the actuator being actuatable to move the wire within the interior channel and into contact with the at least one fastener engagement element to move the at least one fastener engagement element into a fastener engagement position, wherein in the fastener engagement position at least a portion of the at least one fastener engagement element is positioned external to the external periphery of the shank.

2. The apparatus of claim 1, wherein the shank comprises a fastener engagement portion comprising at least one opening in the external periphery, and wherein in the fastener engagement position, the at least one fastener engagement element extends partially through the at least one opening.

3. The apparatus of claim 2, wherein the fastener engagement portion of the shank is engageable with the fastener to form a first coupling between the shank and fastener, and wherein in the fastener engagement position the at least one fastener engagement element is engageable with the fastener to form a second coupling between the shank and the fastener.

4. The apparatus of claim 1, wherein the handle is graspable by a single hand of a user and the actuator is actuatable by said single hand while grasping the handle with said single hand.

5. The apparatus of claim 1, wherein a distal end portion of the wire comprises a beveled surface, the actuator being actuatable to move the wire to contact the at least one fastener engagement element with the beveled surface, wherein the beveled surface promotes outward movement of the at least one fastener engagement element away from the interior channel.

6. The apparatus of claim 1, wherein the at least one fastener engagement element comprises at least one of a ball and pin.

7. The apparatus of claim 1, wherein the at least one fastener engagement element comprises a plurality of fastener engagement elements each separately movable relative to each other.

8. The apparatus of claim 1, wherein the actuator comprises a plunging mechanism, the plunging mechanism comprising a depressible button, wherein the depressible button is depressible a first time to move the wire within the interior channel and into contact with the at least one fastener engagement element to move the at least one fastener engagement element into the fastener engagement position, and depressible a second time to move the wire within the interior channel out of contact with the at least one fastener engagement element to allow the at least one fastener engagement element to move out of the fastener engagement position.

9. The apparatus of claim 1, wherein the actuator comprises a collar translationally movable between first and second positions, wherein in the first position the wire is in contact with the at least one fastener engagement element to move the at least one fastener engagement element into the fastener engagement position, and in the second position the wire is out of contact with the at least one fastener engagement element to allow the at least one fastener engagement element to move out of the fastener engagement position, the collar being biased in the first position.

10. The apparatus of claim 9, wherein the collar is rotationally movable relative to the handle to rotate the shank relative to the handle.

11. The apparatus of claim 1, wherein the actuator comprises a swivel rotationally movable relative to the handle to translationally move the wire between first and second positions, wherein in the first position the wire is in contact with the at least one fastener engagement element to move the at least one fastener engagement element into the fastener engagement position, and in the second position the wire is out of contact with the at least one fastener engagement element to allow the at least one fastener engagement element to move out of the fastener engagement position.

12. The apparatus of claim 1, wherein the shank comprises a first internal shank, the apparatus further comprising a second external shank, wherein the first internal shank is positioned within the second external shank, and the second external shank comprising a plurality of teeth configured to engage the fastener.

13. The apparatus of claim 12, wherein the actuator comprises a first actuator, the apparatus further comprising a second actuator coupled to the shank, the second actuator being actuatable to translationally move the second external shank relative to the first internal shank.

14. A fastener, comprising:
a head comprising a recess for receiving a first portion of a tool, the recess comprising at least one sidewall and at least one indentation formed in the sidewall, the at least one indentation configured to receive a second portion of the tool; and
a shank coupled to the head, the shank comprising external threads.

15. The fastener of claim 14, wherein the at least one indentation comprises a plurality of substantially semi-spherically shaped indentations.

16. The fastener of claim 14, wherein the at least one indentation comprises a single slot extending about the recess.

17. The fastener of claim 14, further comprising a plurality of notches formed in the head and positioned about the recess.

18. The fastener of claim 14, wherein the head comprises a plurality of spaced-apart radially outwardly extending arms configured to radially inwardly collapse under a predetermined pressure threshold, and wherein an outer periphery of each of the arms comprises external threads.

19. A system for installing and removing fasteners, comprising:
a tool comprising a handle and a driver portion coupled to the handle, the driver portion comprising a first fastener engaging element, a shaft translationally movable relative to the handle, and at least one second fastener engaging element outwardly movable in a direction substantially transverse relative to the translational movement of the shaft, wherein the shaft is translationally movable via actuation of an actuator to contact and outwardly move the plurality of second fastener engaging elements; and
a fastener comprising a head and a shank, wherein the head comprises a recess configured to matingly receive the first fastener engaging element of the tool, the recess comprising at least one sidewall and at least one indentation formed in the at least one sidewall, the at least one indentation configured to receive the at least one second fastener engaging element when outwardly moved by the shaft.

20. The system of claim 19, wherein:
the driver portion comprises a plurality of third fastener engaging elements positionable about the first and second fastener engaging elements; and
the fastener further comprising a plurality of notches positioned about the recess, the plurality of notches configured to matingly receive the plurality of third fastener engaging elements.

21. A method for installing fasteners into an object and removing fasteners from an object, comprising:
positioning a first fastener engaging element of a tool into a recess of a fastener; and
with the first fastener engaging element positioned in the recess of the fastener, actuating an actuator of the tool to outwardly move and retain at least one second engaging element of the tool into a corresponding at least one engaging element receiver formed in a sidewall of the recess.

22. The method of claim 21, wherein positioning the first fastener engaging element and actuating the actuator of the tool are performable with one hand.

23. The method of claim 21, further comprising:
with the first fastener engaging element in the recess of the fastener and the at least one second engaging element outwardly moved in the corresponding at least one engaging element receiver, positioning the tool proximate a fastener installation site on the object and rotating the tool in a driving direction to drive the fastener into the object;
with the fastener driven into the object, actuating the actuator of the tool to release the at least one second engaging element from the at least one engaging element receiver; and
with the at least one second engaging element released from the at least one engaging element receiver, removing the first fastener engaging element of the tool out of the recess of the fastener.

24. The method of claim 21, wherein the at least one engaging element receiver comprises at least one first engaging element receiver, the method further comprising:
actuating a second actuator of the tool to move at least one third engaging element of the tool into a corresponding at least one second engaging element receiver formed in the fastener when the fastener is driven in an object; and
with the at least one third engaging element of the tool moved into the corresponding at least one second engaging element receiver formed in the fastener, and with the fastener installed in the object, rotating the tool in a loosening direction.

25. A locking plate system, comprising:
a locking plate comprising a plurality of circular apertures with internal threads, wherein at least one of the apertures comprises a plurality of spaced-apart notches positioned about a periphery of the at least one of the apertures, the plurality of spaced-apart notches defining a plurality of spaced-apart tabs each positioned between adjacent notches, wherein the internal threads of the at least one of the apertures are formed in the plurality of spaced-apart tabs.

26. The locking plate system of claim 25, further comprising circumferentially extending slits formed in the plurality of spaced-apart tabs.

27. The locking plate system of claim 25, further comprising a screw alignment guide comprising a threaded channel for receiving a threaded fastener, the alignment guide further comprising a plurality of tabs sized, shaped, and positioned to matingly engage the plurality of spaced-apart notches of the at least one of the apertures and orient the threaded channel into coaxial alignment with the at least one of the apertures.

28. The locking plate system of claim 27, further comprising a drill guide comprising a channel sized to matingly receive a drill bit, the drill guide being positionable within and in coaxial alignment with the threaded channel of the screw alignment guide.
